(19) United States
(12) Patent Application Publication
(10) Pub. No.: US 2020/0397278 A1
(43) Pub. Date: Dec. 24, 2020

(54) ENDOSCOPE SYSTEM, IMAGE PROCESSING APPARATUS, IMAGE PROCESSING METHOD, AND RECORDING MEDIUM

(71) Applicant: OLYMPUS CORPORATION, Tokyo (JP)

(72) Inventors: Kei KUBO, Tokyo (JP); Makoto IGARASHI, Tokyo (JP)

(73) Assignee: OLYMPUS CORPORATION, Tokyo (JP)

(21) Appl. No.: 17/010,379

(22) Filed: Sep. 2, 2020

Related U.S. Application Data
(63) Continuation of application No. PCT/JP2018/029674, filed on Aug. 7, 2018.

(30) Foreign Application Priority Data
Mar. 5, 2018 (JP) 2018-038793

(51) Int. Cl. A61B 1/313; A61B 1/00; A61B 1/06

(52) U.S. Cl. CPC A61B 1/3137; A61B 1/00045; A61B 1/0684; A61B 1/0676; A61B 1/00009

(57) ABSTRACT

An endoscope system includes a light source apparatus configured to generate illumination light to irradiate an object, an image pickup device configured to pick up an image of the object to output an image pickup signal, and a processor configured to generate first color component data corresponding to first light having a center wavelength within a wavelength range where both respective light absorption coefficients in light absorption characteristics of oxyhemoglobin and deoxyhemoglobin are low and second color component data corresponding to second light having a center wavelength in a blue region or a green region based on the image pickup signal, and assign the first color component data to two out of three channels including blue, green, and red channels of an image display apparatus and assign the second color component data to a remaining one of the three channels, to generate an observation image.

### CROSS REFERENCE TO RELATED APPLICATION

This application is a continuation application of PCT/JP2018/029674 filed on Aug. 7, 2018 and claims benefit of Japanese Application No. 2018-038793 filed in Japan on Mar. 5, 2018, the entire contents of which are incorporated herein by this reference.

### BACKGROUND OF THE INVENTION

### 1. Field of the Invention

The present invention relates to an endoscope system, an image processing apparatus, an image processing method, and a recording medium, and more particularly to an endoscope system, an image processing apparatus, an image processing method, and a recording medium used to observe a living tissue.

### 2. Description of the Related Art

In endoscope observation in the medical field, an observation method of irradiating a living tissue with narrow-band light having a center wavelength (wavelength band) set depending on a light absorption characteristic of hemoglobin to visualize a blood vessel existing at a desired depth of the living tissue has been conventionally proposed.

More specifically, Japanese Patent No. 5427318 discloses a configuration in which a mucous membrane is irradiated with narrow-band light in the vicinity of 600 nm as light that is relatively easy to be absorbed in hemoglobin and narrow-band light in the vicinity of 630 nm as light that is relatively difficult to be absorbed in hemoglobin to display a thick blood vessel existing at a depth of the mucous membrane with high contrast, for example.

In endoscope observation in the medical field, there has occurred a problem that if a situation where at least a part of a surface of an object is covered with blood has occurred, visibility of a region covered with the blood may decrease to such a degree that presence or absence of existence of a tissue other than a mucous membrane cannot be judged, for example.

### SUMMARY OF THE INVENTION

An endoscope system according to an aspect of the present invention includes a light source apparatus configured to generate illumination light to irradiate an object, an image pickup device configured to pick up an image of the object irradiated with the illumination light to output an image pickup signal, and a processor configured to generate first color component data corresponding to first light having a center wavelength within a wavelength range from a red region to a near-infrared region where both respective light absorption coefficients in light absorption characteristics of oxyhemoglobin and deoxyhemoglobin are low and second color component data corresponding to second light having a center wavelength in a blue region or a green region based on the image pickup signal outputted from the image pickup device, and assign the first color component data to two out of three channels including a blue channel, a green channel, and a red channel of an image display apparatus and assign the second color component data to a remaining one of the three channels, to generate a first observation image.

An image processing apparatus according to an aspect of the present invention is an image processing apparatus that processes an image pickup signal generated by picking up an image of an object irradiated with illumination light, the image processing apparatus generating first color component data corresponding to first light having a center wavelength within a wavelength range from a red region to a near-infrared region where both respective light absorption coefficients in light absorption characteristics of oxyhemoglobin and deoxyhemoglobin are low and second color component data corresponding to second light having a center wavelength in a blue region or a green region based on the image pickup signal, and assigning the first color component data to two out of three channels including a blue channel, a green channel, and a red channel of an image display apparatus and assigning the second color component data to a remaining one of the three channels, to generate an observation image.

An image processing method according to an aspect of the present invention is an image processing method for processing an image pickup signal generated by picking up an image of an object irradiated with illumination light, the method including generating first color component data corresponding to first light having a center wavelength within a wavelength range from a red region to a near-infrared region where both respective light absorption coefficients in light absorption characteristics of oxyhemoglobin and deoxyhemoglobin are low and second color component data corresponding to second light having a center wavelength in a blue region or a green region based on the image pickup signal, and assigning the generated first color component data to two out of three channels including a blue channel, a green channel, and a red channel of an image display apparatus and assigning the generated second color component data to a remaining one of the three channels, to generate an observation image.

A recording medium according an aspect of the present invention is a non-transitory computer-readable recording medium storing an image processing program executed by a computer, the image processing program causing an image processing apparatus that processes an image pickup signal generated by picking up an image of an object irradiated with illumination light to perform data generation processing for generating first color component data corresponding to first light having a center wavelength within a wavelength range from a red region to a near-infrared region where both respective light absorption coefficients in light absorption characteristics of oxyhemoglobin and deoxyhemoglobin are low and second color component data corresponding to second light having a center wavelength in a blue region or a green region based on the image pickup signal, and observation image generation processing for assigning the first color component data generated by the data generation processing to two out of three channels including a blue channel, a green channel, and a red channel of an image display apparatus and assigning the second color component data generated by the data generation processing to a remaining one of the three channels, to generate an observation image.

### BRIEF DESCRIPTION OF THE DRAWINGS

FIG. 1 is a diagram illustrating a configuration of a principal part of an endoscope system according to an embodiment;

FIG. 2 is a diagram illustrating an example of a wavelength band of light to be emitted from each of LEDs provided in a light source apparatus in the endoscope system according to the embodiment;

FIG. 3 is a schematic view illustrating an example of an observation image to be displayed when an observation mode of the endoscope system according to the embodiment is set to a white light observation mode;

FIG. 4 is a diagram illustrating respective light absorption characteristics of oxyhemoglobin and deoxyhemoglobin;

FIG. 5 is diagram illustrating a light absorption characteristic of fat; and

FIG. 6 is a schematic view illustrating an example of an observation image to be displayed when the observation mode of the endoscope system according to the embodiment is set to a special light observation mode.

### DETAILED DESCRIPTION OF THE PREFERRED EMBODIMENT

An embodiment of the present invention will be described below with reference to the drawings.

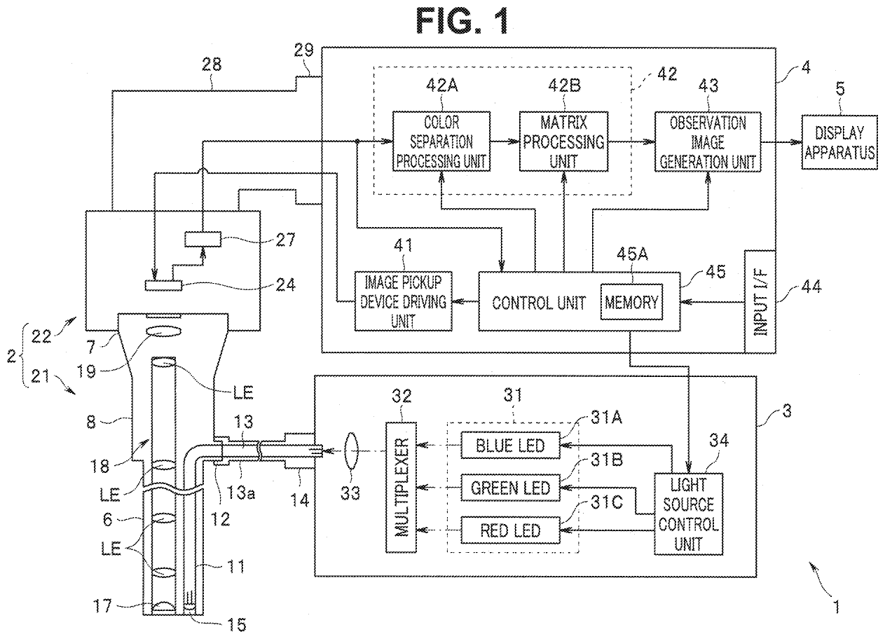

FIGS. 1 to 6 relate to the embodiment of the present invention.

An endoscope system 1 includes an endoscope apparatus 2 configured to be inserted into a subject and output image data obtained by picking up an image of an object such as a living tissue within the subject, a light source apparatus 3 configured to supply illumination light to irradiate the object to the endoscope apparatus 2, a processor 4 configured to generate an observation image based on the image data outputted from the endoscope apparatus 2 and output the generated observation image, and a display apparatus 5 configured to display the observation image outputted from the processor 4 on a screen, as illustrated in FIG. 1. FIG. 1 is a diagram illustrating a configuration of a principal part of the endoscope system according to the embodiment.

The endoscope apparatus 2 includes an optical viewing tube 21 including an elongated insertion section 6 and a camera unit 22 detachably attachable to an eyepiece section 7 in the optical viewing tube 21.

The optical viewing tube 21 includes the elongated insertion section 6 insertable into the subject, a grasping section 8 provided at a proximal end portion of the insertion section 6, and the eyepiece section 7 provided on a proximal end portion of the grasping section 8.

A light guide 11 configured to transmit illumination light supplied via a cable 13a is inserted, as illustrated in FIG. 1, into the insertion section 6.

An emission end portion of the light guide 11 is arranged in the vicinity of an illumination lens 15 in a distal end portion of the insertion section 6, as illustrated in FIG. 1. An incidence end portion of the light guide 11 is arranged in a light guide pipe sleeve 12 provided on the grasping section 8.

A light guide 13 configured to transmit illumination light supplied from the light source apparatus 3 is inserted, as illustrated in FIG. 1, into the cable 13a. A connection member (not illustrated) detachably attachable to the light guide pipe sleeve 12 is provided at one end portion of the cable 13a. A light guide connector 14 detachably attachable to the light source apparatus 3 is provided at the other end portion of the cable 13a.

The distal end portion of the insertion section 6 is provided with the illumination lens 15 configured to emit illumination light transmitted from the light guide 11 to outside and an objective lens 17 configured to obtain an optical image corresponding to light incident from outside. An illumination window (not illustrated) in which the illumination lens 15 is arranged and an objective window (not illustrated) in which the objective lens 17 is arranged are provided adjacent to each other on a distal end surface of the insertion section 6.

A relay lens 18 including a plurality of lenses LE configured to transmit the optical image obtained by the objective lens 17 to the eyepiece section 7 is provided, as illustrated in FIG. 1, in the insertion section 6. In other words, the relay lens 18 is configured to have a function as a transmission optical system configured to transmit light incident from the objective lens 17.

An eyepiece lens 19 configured to be able to observe the optical image transmitted by the relay lens 18 with naked eyes is provided, as illustrated in FIG. 1, within the eyepiece section 7.

The camera unit 22 includes an image pickup device 24 and a signal processing circuit 27. The camera unit 22 is configured to be detachably attachable to the processor 4 via a connector 29 provided at an end portion of a signal cable 28.

The image pickup device 24 is composed of an image sensor such as a color CMOS. The image pickup device 24 is configured to perform an image pickup operation corresponding to an image pickup device driving signal outputted from the processor 4. The image pickup device 24 is configured to have a function as an image pickup unit and pick up an image of light emitted via the eyepiece lens 19 and generate and output an image pickup signal corresponding to the light the image of which has been picked up.

The signal processing circuit 27 is configured to subject the image pickup signal outputted from the image pickup device 24 to predetermined signal processing such as correlated double sampling processing, gain adjustment processing, and A/D conversion processing. The signal processing circuit 27 is configured to output image data obtained by subjecting the image pickup signal to the above-described predetermined signal processing to the processor 4 to which the signal cable 28 is connected.

The light source apparatus 3 is configured to have a function as a light source unit and generate illumination light for illuminating a surface of an object at least a part of which is covered with blood. The light source apparatus 3 includes a light emitting unit 31, a multiplexer 32, a light collecting lens 33, and a light source control unit 34.

The light emitting unit 31 includes a blue LED 31A, a green LED 31B, and a red LED 31C. In other words, anyone of light sources in the light emitting unit 31 is composed of a semiconductor light source.

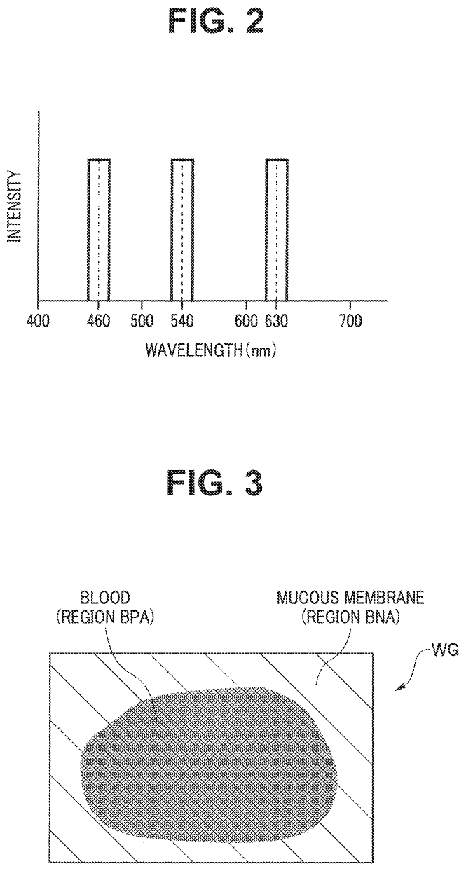

The blue LED 31A is configured to generate B light as (narrow-band) blue light having a center wavelength and an intensity in a blue region. More specifically, the blue LED 31A is configured to generate B light having a center wavelength set to the vicinity of 460 nm and having a bandwidth set to approximately 20 nm, as illustrated in FIG. 2, for example. The blue LED 31A is configured to emit light or quench light in response to an LED driving signal fed from the light source control unit 34. The blue LED 31A is configured to generate B light having a light emission amount corresponding to the LED driving signal fed from the light source control unit 34. FIG. 2 is a diagram illustrating an example of a wavelength band of light emitted from each of the LEDs provided in the light source apparatus in the endoscope system according to the embodiment.

The green LED 31B is configured to generate G light as (narrow-band) green light having a center wavelength and an intensity in a green region. More specifically, the green LED 31B is configured to generate G light having a center wavelength set to the vicinity of 540 nm and having a bandwidth set to approximately 20 nm, as illustrated in FIG. 2, for example. The green LED 31B is configured to emit light or quench light in response to an LED driving signal fed from the light source control unit 34. The green LED 31B is configured to generate G light having a light emission amount corresponding to the LED driving signal fed from the light source control unit 34.

The red LED 31C is configured to generate R light as (narrow-band) red light having a center wavelength and an intensity in a red region. More specifically, the red LED 31C is configured to generate R light having a center wavelength set to the vicinity of 630 nm and having a bandwidth set to approximately 20 nm, as illustrated in FIG. 2, for example. The red LED 31C is configured to emit light or quench light in response to an LED driving signal fed from the light source control unit 34. The red LED 31C is configured to generate R light having a light emission amount corresponding to the LED driving signal fed from the light source control unit 34.

The multiplexer 32 is configured to be able to multiplex the lights emitted from the light emitting unit 31 and make the multiplexed lights incident on the light collecting lens 33.

The light collecting lens 33 is configured to collect the lights incident via the multiplexer 32 and emit the collected lights to the light guide 13.

The light source control unit 34 includes a control circuit, for example. The light source control unit 34 is configured to generate and output an LED driving signal for driving each of the LEDs in the light emitting unit 31 in response to a control signal outputted from the processor 4.

The processor 4 includes an image pickup device driving unit 41, an image processing unit 42, an observation image generation unit 43, an input I/F (interface) 44, and a control unit 45.

The image pickup device driving unit 41 is configured to generate and output an image pickup device driving signal for driving the image pickup device 24 in response to the control signal outputted from the control unit 45.

The image processing unit 42 includes a color separation processing unit 42A and a matrix processing unit 42B.

The color separation processing unit 42A is configured to perform color separation processing for generating, based on the image data outputted from the signal processing circuit 27, a plurality of spectral image data respectively corresponding to a plurality of color components included in the image data, in response to the control signal outputted from the control unit 45. The color separation processing unit 42A is configured to output the plurality of spectral image data obtained as a processing result of the above-described color separation processing to the matrix processing unit 42B.

The matrix processing unit 42B is configured to perform matrix processing for generating image data corresponding to a plurality of color components by using the plurality of spectral image data outputted from the color separation processing unit 42A, in response to the control signal outputted from the control unit 45. The matrix processing unit 42B is configured to output the image data corresponding to the plurality of color components obtained as a processing result of the above-described matrix processing to the observation image generation unit 43.

The observation image generation unit 43 is configured to selectively assign the image data corresponding to the plurality of color components outputted from the matrix processing unit 42B to a B (blue) channel, a G (green) channel, and an R (red) channel of the display apparatus 5 to generate an observation image in response to the control signal outputted from the control unit 45. The observation image generation unit 43 is configured to output the observation image generated as described above to the display apparatus 5.

The input I/F 44 includes one or more switches and/or buttons capable of issuing an instruction or the like corresponding to a user's operation. More specifically, the input I/F 44 includes an observation mode changeover switch (not illustrated) capable of issuing an instruction to set (switch) an observation mode of the endoscope system 1 to either one of a white light observation mode and a special light observation mode in response to a user's operation, for example.

The control unit 45 includes a memory 45A storing control information or the like used in controlling each of the units in the endoscope system 1. The control unit 45 is configured to generate and output a control signal for performing an operation corresponding to the observation mode of the endoscope system 1 based on the instruction issued in the observation mode changeover switch in the input I/F 44. The control unit 45 is configured to generate a control signal for setting an exposure period, a reading period, and the like of the image pickup device 24 and output the generated control signal to the image pickup device driving unit 41. The control unit 45 is configured to generate and output a control signal for controlling an operation of each of the LEDs in the light emitting unit 31 via the light source control unit 34.

The control unit 45 is configured to perform brightness detection processing for detecting a current brightness in the observation mode set in the input I/F 44 based on the image data outputted from the signal processing circuit 27. The control unit 45 is configured to generate a control signal for performing a light adjustment operation to bring the current brightness obtained as a processing result of the above-described brightness detection processing closer to a previously set brightness target value for each of the observation modes settable in the input I/F 44 and output the generated control signal to the light source control unit 34.

Note that in the present embodiment, each of the units other than the input I/F 44 in the processor 4 may be configured as an individual electronic circuit, or may be configured as a circuit block in an integrated circuit such as an FPGA (field programmable gate array). In the present embodiment, the processor 4 may include one or more CPUs, for example. The configuration according to the present embodiment may be appropriately modified so that a program for executing a function of each of the units other than the input I/F 44 in the processor 4 is read from the memory 45A and an operation corresponding to the read program is performed in a computer, for example.

The display apparatus 5 includes an LCD (liquid crystal display), for example, and is configured to be able to display the observation image or the like outputted from the processor 4.

Next, the function of the present embodiment will be described below.

A user such as an operator connects each of the units in the endoscope system 1 and turns on power, and then operates the observation mode changeover switch in the input I/F 44, to issue an instruction to set an observation mode of the endoscope system 1 to a white light observation mode, for example.

The control unit 45 generates a control signal for simultaneously emitting B light, G light, and R light from the light source apparatus 3 and outputs the generated control signal to the light source control unit 34 when detecting that the instruction to set the observation mode of the endoscope system 1 to the white light observation mode is issued. The control unit 45 generates a control signal for performing an operation corresponding to the white light observation mode and outputs the generated control signal to the image pickup device driving unit 41, the image processing unit 42, and the observation image generation unit 43 when detecting that the instruction to set the observation mode of the endoscope system 1 to the white light observation mode is issued.

The light source control unit 34 generates an LED driving signal for simultaneously emitting the blue LED 31A, the green LED 31B, and the red LED 31C in the white light observation mode in response to the control signal outputted from the control unit 45 and outputs the generated LED driving signal to the light emitting unit 31. White light including the Blight, the G light, and the R light is emitted as illumination light from the light source apparatus 3 (the light emitting unit 31) in the white light observation mode in response to such an operation of the light source control unit 34, an object is irradiated with the illumination light, an image pickup signal generated by picking up an image of return light (reflected light) of the illumination light is outputted to the signal processing circuit 27 from the image pickup device 24, and image data generated based on the image pickup signal is outputted to the color separation processing unit 42A from the signal processing circuit 27.

The color separation processing unit 42A performs color separation processing for generating B spectral image data corresponding to a blue component included in the image data, G spectral image data corresponding to a green component included in the image data, and R spectral image data corresponding to a red component included in the image data by using image data outputted from the signal processing circuit 27 at the time of the white light observation mode, in response to the control signal outputted from the control unit 45. The color separation processing unit 42A outputs the B spectral image data, the G spectral image data, and the R spectral image data obtained as a processing result of the above-described color separation processing to the matrix processing unit 42B.

The matrix processing unit 42B performs matrix processing for generating B component image data corresponding to the blue component using the B spectral image data outputted from the color separation processing unit 42A, generating G component image data corresponding to the green component using the G spectral image data outputted from the color separation processing unit 42A, and generating R component image data corresponding to the red component using the R spectral image data outputted from the color separation processing unit 42A, in the white light observation mode, in response to the control signal outputted from the control unit 45. The matrix processing unit 42B outputs the B component image data, the G component image data, and the R component image data obtained as a processing result of the above-described matrix processing to the observation image generation unit 43.

The observation image generation unit 43 assigns the B component image data outputted from the matrix processing unit 42B to a B channel of the display apparatus 5, assigns the G component image data outputted from the matrix processing unit 42B to a G channel of the display apparatus 5, and assigns the R component image data outputted from the matrix processing unit 42B to an R channel of the display apparatus 5 to generate a white light observation image in the white light observation mode, in response to the control signal outputted from the control unit 45. The observation image generation unit 43 outputs the white light observation image generated as described above to the display apparatus 5.

The user inserts the insertion section 6 into a subject while confirming the white light observation image displayed on the display apparatus 5, and arranges the distal end portion of the insertion section 6 in the vicinity of a desired object within the subject. Then, the user operates the observation mode changeover switch in the input I/F 44 to issue an instruction to set the observation mode of the endoscope system 1 to a special light observation mode in a situation where a white light observation image WG as schematically illustrated in FIG. 3 is displayed on the display apparatus 5, for example, as a treatment for the desired object is performed, for example. Note that the white light observation image WG illustrated in FIG. 3 represents an example of a situation where it can be judged that a tissue other than a mucous membrane does not exist in a region BNA corresponding to a region not covered with blood and it cannot be judged whether or not the tissue other than the mucous membrane exists in a region BPA corresponding to a region covered with blood, on a surface of the object an image of which is picked up by the endoscope apparatus 2 (the image pickup device 24). FIG. 3 is a schematic view illustrating an example of an observation image displayed when the observation mode of the endoscope system according to the embodiment is set to the white light observation mode.

The control unit 45 generates a control signal for simultaneously emitting the B light and the R light from the light source apparatus 3 and outputs the generated control signal to the light source control unit 34, for example, when detecting that the instruction to set the observation mode of the endoscope system 1 to the special light observation mode is issued. The control unit 45 generates a control signal for performing an operation corresponding to the special light observation mode and outputs the generated control signal to the image pickup device driving unit 41, the image processing unit 42, and the observation image generation unit 43 when detecting that the instruction to set the observation mode of the endoscope system 1 to the special light observation mode is issued.

The light source control unit 34 generates an LED driving signal for simultaneously emitting the blue LED 31A and the red LED 31C while quenching the green LED 31B and outputs the generated LED driving signal to the light emitting unit 31 in the special light observation mode in response to the control signal outputted from the control unit 45. Mixed light including the B light and the R light is emitted as illumination light from the light source apparatus 3 (the light emitting unit 31), the object is irradiated with the illumination light, an image pickup signal generated by picking up an image of return light (reflected light) of the illumination light is outputted to the signal processing circuit 27 from the image pickup device 24, and image data generated based on the image pickup signal is outputted to the color separation processing unit 42A from the signal processing circuit 27 in the special light observation mode in response to such an operation of the light source control unit 34.

The color separation processing unit 42A performs color separation processing to generate B spectral image data corresponding to a blue component included in the image data and R spectral image data corresponding to a red component included in the image data by using the image data outputted from the signal processing circuit 27 at the time of the special light observation mode, in response to the control signal outputted from the control unit 45. The color separation processing unit 42A outputs the B spectral image data and the R spectral image data obtained as a processing result of the above-described color separation processing to the matrix processing unit 42B.

The matrix processing unit 42B performs matrix processing to generate B component image data by applying the B spectral image data outputted from the color separation processing unit 42A to the following equation (1) and to generate G component image data and R component image data by applying the R spectral image data outputted from the color separation processing unit 42A to the following equation (1), for example, in the special light observation mode, in response to the control signal outputted from the control unit 45. The matrix processing unit 42B outputs the B component image data, the G component image data, and the R component image data obtained as a processing result of the above-described matrix processing to the observation image generation unit 43.

$$( \begin{matrix}{B}_{out} \\ {G}_{out} \\ {R}_{out}\end{matrix} ) = ( \begin{matrix}1 & 0 \\ 0 & α \\ 0 & β\end{matrix} ) ( \begin{matrix}{B}_{in} \\ {R}_{in}\end{matrix} ) \qquad ( 1 )$$

Note that in the right side of the foregoing equation (1), Bᵢₙ represents a luminance value of one pixel included in the B spectral image data, Rᵢₙ represents a luminance value of corresponding one pixel included in the R spectral image data, and α and β respectively represent constants set to values larger than zero. In the left side of the foregoing equation (1), Bₒᵤₜ represents a luminance value of one pixel included in the B component image data, Gₒᵤₜ represents a luminance value of corresponding one pixel included in the G component image data, and Rₒᵤₜ represents a luminance value of corresponding one pixel included in the R component image data. Description is made below by taking a case where α=β=1 is set as an example unless otherwise specified.

The observation image generation unit 43 assigns the B component image data outputted from the matrix processing unit 42B to the B channel of the display apparatus 5, assigns the G component image data outputted from the matrix processing unit 42B to the G channel of the display apparatus 5, and assigns the R component image data outputted from the matrix processing unit 42B to the R channel of the display apparatus 5 to generate special light observation image in the special light observation mode in response to the control signal outputted from the control unit 45. The observation image generation unit 43 outputs the special light observation image generated as described above to the display apparatus 5.

In other words, according to the above-described operation, the image processing unit 42 generates R component image data corresponding to R light having a center wavelength in the vicinity of 630 nm and B component image data corresponding to B light having a center wavelength in the vicinity of 460 nm based on the image data generated by the signal processing circuit 27 in response to the image pickup signal outputted from the image pickup device 24 in the special light observation mode. According to the above-described operation, the image processing unit 42 generates G component image data and R component image data using the R spectral image data generated based on the image data outputted from the signal processing circuit 27 and generates B component image data using the B spectral image data generated based on the image data in the special light observation mode.

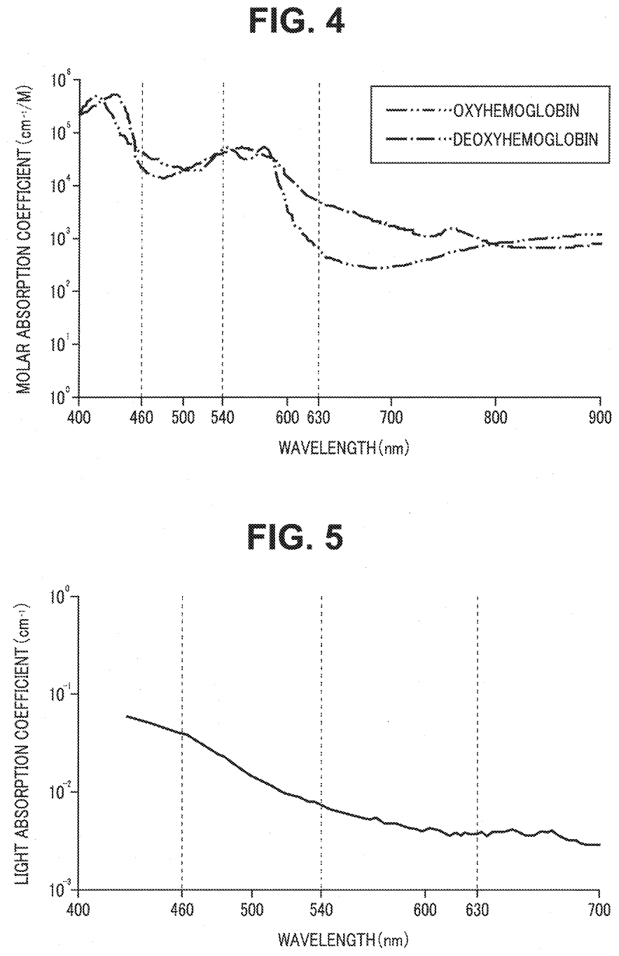

R light included in illumination light to irradiate the object at the time of the special light observation mode can be substantially permeable to blood existing in the region BPA to reach a depth below the surface of the object (a deep layer of a living tissue) because the R light has a center wavelength within a wavelength range where both respective light absorption coefficients in light absorption characteristics of oxyhemoglobin and deoxyhemoglobin are low (see FIG. 4) and a scattering coefficient in a scattering characteristic of the living tissue is low. In other words, in the special light observation mode, when the object is irradiated with illumination light including R light that is high in permeability to blood and is not easily scattered in the living tissue, return light (reflected light) including information about the depth below the surface of the object in the region BPA can be generated. According to the operation of each of the units as described above, in the special light observation mode, the object is irradiated with illumination light including R light to acquire R spectral image data, and a luminance value of the acquired R spectral image data is used as two color components (a green component and a red component) among three color components included in a special light observation image. FIG. 4 is a diagram illustrating respective light absorption characteristics of oxyhemoglobin and deoxyhemoglobin.

B light included in illumination light to irradiate the object at the time of the special light observation mode has a center wavelength within a wavelength range where both respective light absorption coefficients in light absorption characteristics of oxyhemoglobin and deoxyhemoglobin are high (see FIG. 4) and a scattering coefficient in a scattering characteristic of a living tissue is higher than the scattering coefficient of the R light. In other words, in the special light observation mode, when the object is irradiated with illumination light including B light that is easily absorbed by blood and is easily scattered in the living tissue, return light (reflected light) including information about the surface of the object in the region BNA can be generated. B light included in illumination light to irradiate the object at the time of the special light observation mode has a center wavelength within a wavelength range where a light absorption coefficient in a light absorption characteristic of fat is higher than the light absorption coefficient of the R light (see FIG. 5). FIG. 5 is a diagram illustrating a light absorption characteristic of fat.

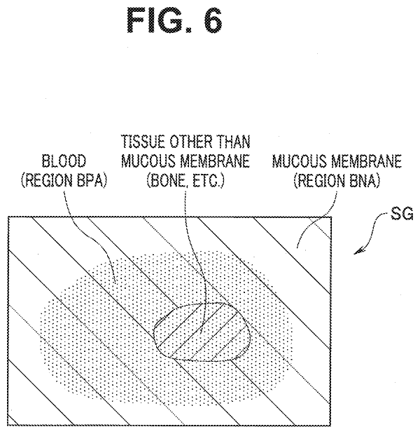

Therefore, according to the present embodiment, in a situation where the white light observation image WG illustrated in FIG. 3 is displayed on the display apparatus 5, when the observation mode of the endoscope system 1 is set to the special light observation mode, a special light observation image SG in which existence of a tissue other than a mucous membrane (a bone, etc.) in the region BPA can be visually recognized, as schematically illustrated in FIG. 6, can be displayed on the display apparatus 5. According to the present embodiment, in the special light observation mode, a special light observation image in which a region where fat exists is indicated with a color tone (e.g., yellow color tone) different from a color tone of the other region can be displayed on the display apparatus 5. FIG. 6 is a schematic view illustrating an example of an observation image displayed when the observation mode of the endoscope system according to the embodiment is set to the special light observation mode.

As described above, according to the present embodiment, in the special light observation mode, a special light observation image having visibility enabling judgment whether or not a tissue other than a mucous membrane exists in a region covered with blood on the surface of the object and capable of identifying a region where fat exists can be displayed. Accordingly, according to the present embodiment, a burden on an operator who performs work with at least a part of the surface of the object covered with blood can be reduced.

According to investigation by the applicant, a finding that a lower limit of a wavelength range where both respective light absorption coefficients of oxyhemoglobin and deoxyhemoglobin are low exists in the vicinity of 615 nm has been obtained. Accordingly, according to the present embodiment, the red LED 31C configured to generate R light having a center wavelength of 615 nm or more may be provided in the light source apparatus 3. Alternatively, in the present embodiment, a near-infrared LD (laser diode) that generates near-infrared light having a center wavelength of 800 nm or less, for example, may be provided in the light source apparatus 3. In other words, the light source apparatus 3 according to the present embodiment may be configured such that light having a center wavelength within a wavelength range from a red region to a near-infrared region where both respective light absorption coefficients in light absorption characteristics of oxyhemoglobin and deoxyhemoglobin are low is generated in the special light observation mode.

In the present embodiment, the image processing unit 42 may be configured to generate two out of three color components including a blue component, a green component, and a red component included in a special light observation image by using the R spectral image data generated based on the image data outputted from the signal processing circuit 27 and generate the remaining one of the three color components included in the special light observation image by using the B spectral image data generated based on the image data. More specifically, the image processing unit 42 may be configured to generate B component image data and R component image data by using the R spectral image data generated based on the image data outputted from the signal processing circuit 27 and generate G component image data by using the B spectral image data generated based on the image data, for example, in the special light observation mode. Alternatively, the image processing unit 42 may be configured to generate B component image data and G component image data by using the R spectral image data generated based on the image data outputted from the signal processing circuit 27 and generate R component image data by using the B spectral image data generated based on the image data, for example, in the special light observation mode.

According to the present embodiment, light to irradiate the object together with R light may be selectable from B light and G light in the special light observation mode. Further, according to the present embodiment, when the object is irradiated with illumination light including R light and G light in the special light observation mode, two out of three color components including a blue component, a green component, and a red component included in a special light observation image may be generated by using R spectral image data, and the remaining one of the three color components included in the special light observation image may be generated by using G spectral image data instead of B spectral image data.

According to the present embodiment, processing for making a ratio of the red component to a total of the color components included in the special light observation image larger than a ratio of the green component to the total of the color components may be performed in the matrix processing unit 42B. More specifically, matrix processing may be performed with respective values of α and β included in a matrix of 3 columns by 2 rows on the right side of the foregoing equation (1) respectively set to values (e.g., α=0.6 and β=1) satisfying a relationship of α<β, for example. According to such setting, it can be judged whether or not a tissue other a mucous membrane exists in a region covered with blood on the surface of the object, and a special light observation image that is high in color reproducibility in a region including blood of the object can be displayed on the display apparatus 5.

According to the present embodiment, 9-axial color correction processing as processing for converting the B component image data, the G component image data, and the R component image data outputted from the matrix processing unit 42B at the time of the special light observation mode into points on a predetermined color space defined by nine reference axes respectively corresponding to predetermined nine hues (magenta, blue, blue cyan, cyan, green, yellow, red yellow, red, and red magenta) and correcting the image data may be performed in the image processing unit 42, for example. Note that in such a case, the B component image data, the G component image data, and the R component image data obtained as a processing result of the above-described 9-axial color correction processing may be outputted to the observation image generation unit 43.

According to the present embodiment, the G component image data and the R component image data outputted from the matrix processing unit 42B at the time of the special light observation mode may be each subjected to structure enhancement processing as processing for applying a spatial filter such as edge enhancement in the image processing unit 42, for example. Note that in such a case, an operation for assigning the B component image data outputted from the matrix processing unit 42B to the B channel of the display apparatus 5, assigning the G component image data obtained as a processing result of the above-described structure enhancement processing to the G channel of the display apparatus 5, and assigning the R component image data obtained as a processing result of the above-described structure enhancement processing to the R channel of the display apparatus 5 may be performed in the observation image generation unit 43, for example.

According to the present embodiment, a dichroic prism configured to separate light emitted via the eyepiece lens 19 into light in three wavelength bands, i.e., light in a blue region, light in a green region, and light in a red region to a near-infrared region and emit the lights and three image pickup devices configured to respectively pick up images of the lights in the three wavelength bands emitted via the dichroic prism may be provided in the camera unit 22, for example, instead of the image pickup device 24.

According to the present embodiment, the image pickup device 24 may be composed of a monochrome image sensor, for example. Note that in such a case, a control signal for emitting B light, G light, and R light from the light source apparatus 3 by time division (sequentially) may be outputted to the light source control unit 34 from the control unit 45 in the white light observation mode, for example. In the above-described case, a control signal for emitting B light and R light from the light source apparatus 3 by time division (alternately) may be outputted to the light source control unit 34 from the control unit 45 in the special light observation mode, for example.

According to the present embodiment, white light in a broader band than a band of light obtained by mixing B light. G light, and R light may be used as illumination light to irradiate the object in the special light observation mode, for example. Note that in such a case, return light from the object may be separated into B light. G light, and R light in the image pickup device 24.

According to the present embodiment, spectral estimation processing for estimating and acquiring R spectral image data by applying a predetermined spectral estimation matrix to the B image data outputted from the signal processing circuit 27 in individually irradiating the object with B light may be performed as processing of the image processing unit 42 in the special light observation mode, for example. Note that in such a case, the color separation processing unit 42A is not required. Accordingly, the B image data outputted from the signal processing circuit 27 and the R spectral image data obtained as a processing result of the above-described spectral estimation processing may be each outputted to the matrix processing unit 42B.

According to the present embodiment, spectral estimation processing for estimating and acquiring B spectral image data by applying a predetermined spectral estimation matrix to the R image data outputted from the signal processing circuit 27 in individually irradiating the object with R light may be performed as processing of the image processing unit 42 in the special light observation mode, for example. Note that in such a case, the color separation processing unit 42A is not required. Accordingly, the R image data outputted from the signal processing circuit 27 and the B spectral image data obtained as a processing result of the above-described spectral estimation processing may be each outputted to the matrix processing unit 42B.

According to the present embodiment, the light source apparatus 3 (the light emitting unit 31) may generate light including B light, G light, and R light as illumination light, the color separation processing unit 42A may generate B spectral image data, G spectral image data, and R spectral image data based on the image data outputted from the signal processing circuit 27, the matrix processing unit 42B may generate color components respectively included in a white light observation image and a special light observation image by using the B spectral image data, the G spectral image data, and the R spectral image data, and the observation image generation unit 43 may display the white light observation image and the special light observation image together on the display apparatus 5, for example. Note that in such a case, a white light observation image may be generated by applying respective operations of the image processing unit 42 and the observation image generation unit 43 at the time of the white light observation mode, and a special light observation image may be generated by applying respective operations of the image processing unit 42 and the observation image generation unit 43 at the time of the special light observation mode, for example.

The present invention is not limited to the above-described embodiment, but it goes without saying that various modifications and applications are possible without departing from the scope and spirit of the invention.

What is claimed is:

1. An endoscope system comprising:
a light source apparatus configured to generate illumination light to irradiate an object;
an image pickup device configured to pick up an image of the object irradiated with the illumination light to output an image pickup signal; and
a processor configured to generate first color component data corresponding to first light having a center wavelength within a wavelength range from a red region to a near-infrared region where both respective light absorption coefficients in light absorption characteristics of oxyhemoglobin and deoxyhemoglobin are low and second color component data corresponding to second light having a center wavelength in a blue region or a green region based on the image pickup signal outputted from the image pickup device, and assign the first color component data to two out of three channels including a blue channel, a green channel, and a red channel of an image display apparatus and assign the second color component data to a remaining one of the three channels, to generate a first observation image.

2. The endoscope system according to claim 1, wherein
the processor assigns the first color component data to the green channel and the red channel and assigns the second color component data to the blue channel.

3. The endoscope system according to claim 2, wherein
the processor performs processing for making a ratio of a red component in the first observation image larger than a ratio of a green component in the first observation image.

4. The endoscope system according to claim 2, wherein
the processor further subjects the first color component data respectively assigned to the green channel and the red channel to structure enhancement processing.

5. The endoscope system according to claim 1, wherein
the light source apparatus generates light including the first light and the second light as the illumination light.

6. The endoscope system according to claim 1, further comprising
a special light observation mode for generating the first observation image as a special light observation image, and
a white light observation mode for radiating white light as the illumination light and generating a second observation image as a white light observation image.

7. The endoscope system according to claim 6, wherein
the light source apparatus
generates light including the first light and the second light having a center wavelength in a blue region as the illumination light in the special light observation mode, and
generates light including the first light, the second light having the center wavelength in the blue region, and third light having a center wavelength in a green region as the illumination light in the white light observation mode, and
the processor
generates the first color component data, the second color component data, and third color component data corresponding to the third light based on an image pickup signal outputted from the image pickup device in the white light observation mode, and
assigns the first color component data to the red channel, assigns the second color component data to the blue channel, and assigns the third color component data to the green channel, to generate the second observation image.

8. The endoscope system according to claim 6, wherein
the light source apparatus
generates light including the first light, the second light having a center wavelength in a blue region, and third light having a center wavelength in a green region as the illumination light, and
the processor
generates the first color component data, the second color component data, and third color component data corresponding to the third light based on an image pickup signal outputted from the image pickup device,
assigns the first color component data to the green channel and the red channel and assigns the second color component data to the blue channel, to generate the first observation image, and
assigns the first color component data to the red channel, assigns the second color component data to the blue channel, and assigns the third color component data to the green channel, to generate the second observation image.

9. The endoscope system according to claim 7, wherein
the light source apparatus generates the first light having a center wavelength set in a vicinity of 630 nm, generates the second light having the center wavelength set in a vicinity of 460 nm, and generates the third light having the center wavelength set in a vicinity of 540 nm.

10. An image processing apparatus that processes an image pickup signal generated by picking up an image of an object irradiated with illumination light, the image processing apparatus
generating first color component data corresponding to first light having a center wavelength within a wavelength range from a red region to a near-infrared region where both respective light absorption coefficients in light absorption characteristics of oxyhemoglobin and deoxyhemoglobin are low and second color component data corresponding to second light having a center wavelength in a blue region or a green region based on the image pickup signal, and assigning the first color component data to two out of three channels including a blue channel, a green channel, and a red channel of an image display apparatus and assigning the second color component data to a remaining one of the three channels, to generate an observation image.

11. An image processing method for processing an image pickup signal generated by picking up an image of an object irradiated with illumination light, the method comprising:
generating first color component data corresponding to first light having a center wavelength within a wavelength range from a red region to a near-infrared region where both respective light absorption coefficients in light absorption characteristics of oxyhemoglobin and deoxyhemoglobin are low and second color component data corresponding to second light having a center wavelength in a blue region or a green region based on the image pickup signal; and
assigning the generated first color component data to two out of three channels including a blue channel, a green channel, and a red channel of an image display apparatus and assigning the generated second color component data to a remaining one of the three channels, to generate an observation image.

12. A non-transitory computer-readable recording medium storing an image processing program executed by a computer,
the image processing program causing an image processing apparatus that processes an image pickup signal generated by picking up an image of an object irradiated with illumination light to perform:
data generation processing for generating first color component data corresponding to first light having a center wavelength within a wavelength range from a red region to a near-infrared region where both respective light absorption coefficients in light absorption characteristics of oxyhemoglobin and deoxyhemoglobin are low and second color component data corresponding to second light having a center wavelength in a blue region or a green region based on the image pickup signal; and
observation image generation processing for assigning the first color component data generated by the data generation processing to two out of three channels including a blue channel, a green channel, and a red channel of an image display apparatus and assigning the second color component data generated by the data generation processing to a remaining one of the three channels, to generate an observation image.
